(19) United States
(12) Patent Application Publication
(10) Pub. No.: US 2017/0258357 A1
(43) Pub. Date: Sep. 14, 2017

(54) INTELLIGENT ELECTRODE

(71) Applicants: Markus RIEMENSCHNEIDER, Taunusstein (DE); Jurgen SAUERZAPF, Mainz (DE)

(72) Inventors: Markus RIEMENSCHNEIDER, Taunusstein (DE); Jurgen SAUERZAPF, Mainz (DE)

(21) Appl. No.: 15/310,111

(22) PCT Filed: Jun. 11, 2015
(86) PCT No.: PCT/EP2015/001179

§ 371 Date: Nov. 10, 2016

(30) Foreign Application Priority Data
Jun. 13, 2014 (DE) 10 2014 008 684.4

(51) Int. Cl. A61B 5/0492

(52) U.S. Cl. CPC A61B 5/0492; A61B 2560/0276; A61B 2562/08

(57) ABSTRACT

The invention relates to a medical electrode for recording bioelectrical signals from a muscle, in particular a cardiac muscle, via the skin of a human or animal. The medical electrode comprises: a metal contact (2) for connection to an electrode cable; an electrically conductive electrode plate (3) for receiving the bioelectrical signals; a contact means (4) for establishing an electrical contact between the electrode plate and the skin; and an electronic circuit (7) which comprises a memory (6) for storing electrode-related data.

The present invention relates in a general manner to a medical electrode for recording bioelectrical signals.

The recording of bioelectrical signals from humans and animals by medical electrodes is known. Bioelectrical signals are produced during muscle activity by the muscle fibers such as, for example, during the activity of a cardiac muscle. These bioelectrical signals can be measured by medical electrodes which are applied on certain points of the body, e.g. on the skin and are transferred via electrode cable to an appropriate device, e.g. an electrocardiogram device. A physician can determine from the course of the bioelectrical signals, which forms, for example, an electrocardiogram or an electromyogram, whether a deviation in the signal course is present and whether the cause of the deviation is based on a disease of the patient.

In order to measure bioelectrical signals such as, e.g. EKG signals, so-called adhesive electrodes are frequently used. Known adhesive electrodes typically have a metallic head for fastening an electrode cable (e.g., clamp or clip), an adhesive surface for adhering the electrode on the patient's skin, a metallic surface as a conductive electrode surface (can form a unit with the metallic head) and a gel for producing the conductive connection between the metallic surface and the skin at the position where the electrode is adhered. Furthermore, such adhesive electrodes often have a protection against drying out which covers the adhesive surface and therefore protects it from drying out.

The gel of such known adhesive electrodes can be subjected to an ageing process since it can, e.g. dry out or chemically change after a certain time. Furthermore, the adhesiveness of the adhesive used with which the adhesive electrode is adhered on the surface of the skin can become weaker. Therefore, it is known that adhesive electrodes can have an expiration date which is printed, e.g., on the packaging of the adhesive electrode.

Moreover, it is known to determine the conductivity of the adhesive electrode as well as the transitional resistance to the skin by an impedance measuring that is integrated in an electrode amplifier and carried out by the latter. It is known for the impedance measuring to feed a high-frequency signal via the electrodes into the body of the patient and to determine the impedance which is characteristic for the conductivity of the adhesive electrode since the impedance forms a measure for the quality of the signal. Furthermore, it is known that a physician visually evaluates the quality of the EKG signal without having an objective criterion available for the signal quality, in particular when no impedance measuring is available.

In particular when using electrodes in a domestic environment the quality of the bioelectrical signal measured with the electrodes becomes especially significant since typically no physician can examine the signal quality there using the EKG. Furthermore, it can be determined by an impedance measuring that the signal quality is poor but the reason for a poor impedance and the associated poor signal quality cannot be determined in this case.

However, there can be many causes for the poor signal quality, wherein the medical electrode and/or its state can have a decisive influence on the signal quality and the impedance. Therefore, e.g. an old gel and therefore, e.g., a chemically changed gel, a dry gel or a poor adhesive connection which occurs in particular when several electrodes are used, can be the cause for a poor electrical conductivity between the electrode and the skin surface and the associated poor impedance and signal quality. Furthermore, e.g. skin care agents such as cremes or also hairs or the nature of the skin itself can decisively influence the line resistance between the skin and the electrode. However, this cannot be readily determined by a user, in particular by lay persons in a domestic environment.

Furthermore, it cannot be determined with the above-described, known electrodes and devices what type of electrode was used for the measuring, e.g., of an EKG. There are various types of electrodes which have different properties and which also can have a decisive influence on the signal quality. The shape factor of the electrode as well as the gel play a part here. Large electrodes with a correspondingly greater amount of gel typically have a good conductivity but a poor local specificity. In contrast thereto, smaller electrodes have, due to a lesser amount of gel, a poorer conductivity but therefore a greater local specificity due to the lesser expansion.

Furthermore, there are also so-called dry electrodes which have instead of gel a conductive, gel-like pad which produces the contact with the skin.

Furthermore, the gel can have such a nature that it very rapidly establishes a good electrical conductivity with the skin, wherein such gels are typically very aggressive and attack the skin. On the other hand, long-time electrodes are known in which the gel is designed to be less aggressive on account of the longer wearing time of the electrodes, which can, however, result in that a sufficient conductivity to the skin is established only after a rather long time, e.g. several minutes.

Therefore, the exact knowledge about the above-cited details of the electrode can be important for determining the cause of a poor signal quality. However, this can be difficult with the known electrodes since this information cannot always be gained from the medical electrode itself but rather, e.g., is printed only on a packaging in which the medical electrode is packed.

The present invention has the problem of making an improved electrode available which at least partially overcomes the above-cited disadvantages of the prior art.

This problem is solved by the subject matter of claim 1.

A medical electrode according to the invention for recording bioelectrical signals of a muscle, in particular of a cardiac muscle, via the skin of a person or of an animal comprises:

a metallic contact for the connection to an electrode cable;

an electrically conductive electrode plate for receiving the bioelectrical signals;

a contact means for establishing an electrical contact between the electrode plate and the skin; and

an electronic circuit comprising a memory for storing electrode-related data.

Other aspects and features of the present invention result from the dependent claims, the attached drawings and the following description of preferred exemplary embodiments.

The exemplary embodiments relate to a medical electrode for recording bioelectrical signals of a muscle, in particular of a cardiac muscle, via the skin of a person or of an animal. The medical electrode is also designated only as electrode in the following. The electrode comprises a metallic contact for the connection to an electrode cable, an electrically conductive electrode plate for receiving the bioelectrical signals, a contact means, especially a gel for establishing an electrical contact between the electrode plate and the skin and an electronic circuit which comprises a memory for storing electrode-related data.

In addition, a conductive, gel-like pad which establishes the contact can be provided as contact means instead of a gel in so-called dry electrodes.

The metallic contact can be constructed as a metallic head which for its part is formed as a clip or a clamp or some other clamping means. The metallic head itself can be formed from metal or some other conductive material (e.g., conductive plastic) or can comprise an electrically conductive coating which consists, e.g. of metal or graphite or the like.

The contact means can be present in liquid or gel-like form. However, it is also solid in some exemplary embodiments. The contact means can also have a spongey structure or the like in which, e.g., a contact liquid or a gel is received.

The electrically conductive electrode plate can be designed to be very thin or even be deposited by evaporation. In some exemplary embodiments it is also a component of the metallic contact itself and can, e.g., be a surface of the metallic contact. In some exemplary embodiments the metallic contact, the contact means and/or the electrode plate are constructed in one part or are obtainable in one piece.

Electrode-related data can comprise or represent, e.g., the manufacturer of the electrode, the electrode type, a charge number or series number, type of the contact means, a contact means amount and/or an expiration date or the like.

It is therefore possible to determine whether the correct electrode type was used and, e.g., whether there is a poor signal quality on a poor electrical connection with the skin. It is possible by reading out the electrode-related data to objectively determine whether the poor signal quality is due to an expired expiration date and a correspondingly aged contact means, to a false electrode type or to a short acting time of the contact means (e.g., of the gel) or, e.g., to a production error.

The memory can be designed as a write-read memory and can be, e.g., a flash memory or the like which permanently stores data without a current supply.

In some exemplary embodiments the medical electrode is also designed as a so-called suction electrode in which, e.g., a transitional resistance to the skin of the patient and/or the chemical composition, e.g., of the contact means is/are measured (see also the following description).

In some exemplary embodiments the electronic circuit is set up to transfer the electrode-related data to a receiver. This can take place, e.g., via an electrode cable connected to the electrode. The data can be transferred to an external device as receiver which, e.g., displays or evaluates the data. In some exemplary embodiments the data is also transferred to the device, e.g., an electrocardiogram device, which records the EKG, or to an electromyogram device which records an electromyogram. In some exemplary embodiments the electrode-related data is also transferred to an analysis device which analyses the received electrode-related data and, based on the analysis, emits a reason for a poor signal quality.

In some exemplary embodiments the electrode-related data is only read out of the electrode when an analysis of the received electrode signals showed that a poor signal quality is present. This can also take place automatically in some exemplary embodiments, e.g., in that the EKG device or the electromyogram device to which the electrode is connected is sending a corresponding control signal to the electronic circuit, wherein the latter transfers the electrode-related data in reaction to the received control signal.

In some exemplary embodiments the electronic circuit is designed to transfer the electrode-related data wirelessly, e.g., via inductive or capacitive coupling, by radio or the like. The electronic circuit can comprise a radio module. In some exemplary embodiments the electronic circuit comprises a transponder (e.g., RFID transponder, Engl.: radio-frequency identification) which is designed to wirelessly transmit by radio the electrode-related data in reaction to a received radio signal.

This makes it possible to transmit the electrode-related data without having to connect the electrode to a cable. In some exemplary embodiments electrical energy is also transmitted wirelessly by inductive or capacitive coupling.

In some exemplary embodiments the medical electrode furthermore comprises an electrode body in which the electronic circuit is arranged, wherein electrical contacts are run on an upper side of the electrode body from the electronic circuit. As a result, the electronic circuit of the medical electrode can be contacted from the outside by an electrode plug which contacts the electrical contacts on the upper side of the electrode body in an appropriate manner. In this manner data from the electronic circuit can be transmitted via the electrical contacts and via the electrode plug and/or the electronic circuit can be supplied with electrical energy via the electrode plug.

Furthermore, in some exemplary embodiments the medical electrode comprises a covering on the contact means and comprises a presence sensor. The presence sensor is designed to recognize whether the covering is present. The presence sensor can be formed by two electrodes. After the removal of the covering, e.g., the contact means can spread out and in this manner establishes a current flow between the electrodes by means of which the presence sensor can recognize the removed covering.

In some exemplary embodiments the covering comprises a contact area which makes contact with the two electrodes when the covering is arranged on the medical electrode. According to this, a current flows between the two electrodes which is interrupted when the covering is removed. The presence sensor can recognize using the current interruption that the covering was removed.

In some exemplary embodiments the medical electrode furthermore comprises a conductivity sensor which is designed for measuring the electrical conductivity of the contact means. As a result, it is possible to determine whether a poor signal quality is due to the contact means, that is, for example, to the gel used. The conductivity of the contact means is not only a function of the type of the contact means, that is, e.g., of the type of gel used but also of how, as explained above, of whether the contact means is aged and/or dried out. An objective parameter can be obtained by the measuring of the conductivity of the contact means which indicates to what extent the contact means is electrically conductive. The measured value of the electrical conductivity can be stored in the memory of the electronic circuit as electrode-related data. Conductivity sensors are basically known. The conductivity sensor can, e.g., comprise two electrodes arranged at a distance from one another so that contact means are located between them. Accordingly, a current flows between the two electrodes through the contact means so that the conductivity of the contact means can be determined.

In some exemplary embodiments the medical electrode according to one of the previous claims furthermore comprises a conductivity sensor which is designed to measure the conductivity between the electrode plate and the skin. The conductivity measuring can in particular also comprise an impedance measuring such as was already described above. To this end the medical electrode comprises an electrode arranged at a distance from the electrode plate so that a current, in particular an alternating current flowing between the electrode plate and the electrode flows through the skin so that the impedance can be determined.

Such a conductivity measuring can also be used in some exemplary embodiments in order to obtain information about the status of an adhesive layer of the medical electrodes with which it is adhered to the skin, or information about the adhesive connection of the medical electrode to the skin.

In some exemplary embodiments the medical electrode furthermore comprises a chemical sensor designed for determining a chemical property of the contact means. Chemical sensors are basically known and can make use of different principles for determining different chemical properties of the contact means. Chemical sensors can make use, e.g., of molecular properties for the detection such as, e.g., a molecular mass, diffusion behavior, molecular structure (magnetic properties, e.g., para-magnetism), molecular stability (bonding energy) and molecular mobility. Chemical properties such as reactivity, ability to be oxidized and reducibility can also be used.

In some exemplary embodiments the electrical circuit is designed to store measuring data from a sensor of the medical electrode in the memory. The sensor here is one of the above-cited sensors or even more than one of the above-cited sensors can be present in the medical electrode. Moreover, the above-cited microcontroller can be designed to perform the method of operation of one or more of the above-cited sensors.

Consequently, in some exemplary embodiments the following influencing factors for the electrode quality and therefore for the signal quality can be at least partially determined:
Using the correct electrode type from the correct producer
Using the electrode within the expiration date
Excluding multiple use of an electrode
The electrode has a sufficient transition resistance for the skin
The electrode has a sufficiently conductive contact means (gel)
The electrode has a gel which is unchanged chemically and biologically.

Consequently, in some exemplary embodiments the signal quality can be objectively determined, outputted and/or stored. The reason for a poor signal quality can be objectively determined, outputted and/or stored. In the case of erroneous measurements which can be traced back to a poor signal quality the reason for this erroneous measurement can be objectively determined, outputted and/or stored and consequently serve as proof for the cause of the erroneous measurement. Moreover, the use of non-admissible electrodes can be objectively determined, outputted and/or stored, as a result of which a proof of this cause for erroneous measurements and a commercial binding of patents to certain medical electrodes are possible. The using of the correct electrode for the correct application can also be determined in many exemplary embodiments, as a result of which a proof of this cause in the case of erroneous measurements and a commercial binding of patents are also possible.

Exemplary embodiments of the invention will now be described by way of example and with reference made to the attached drawings, in which:

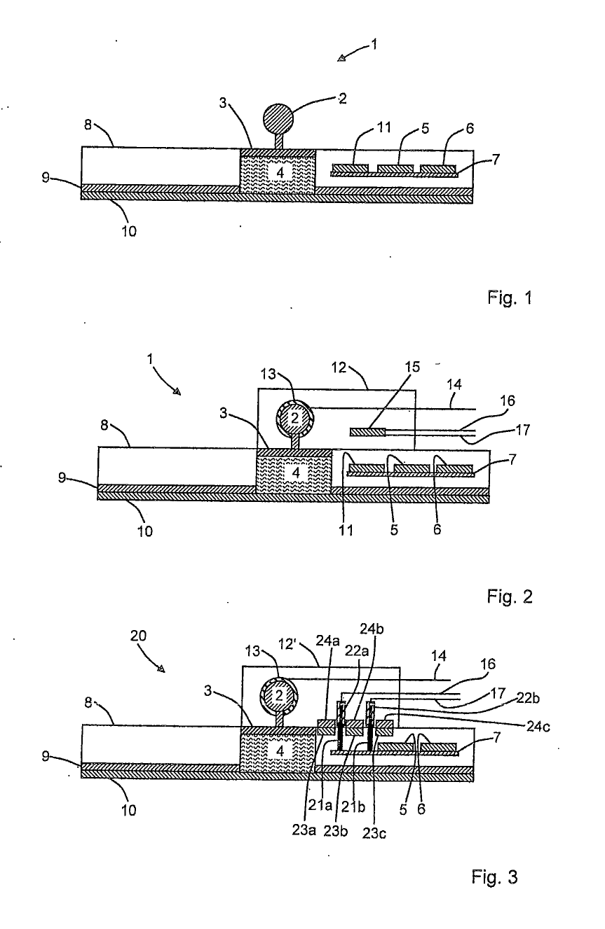

FIG. 1 illustrates a first exemplary embodiment of a medical electrode according to the present invention;

FIG. 2 shows the medical electrode of FIG. 1 with an electrode plug;

FIG. 3 illustrates a second exemplary embodiment of a medical electrode with another electrode plug;

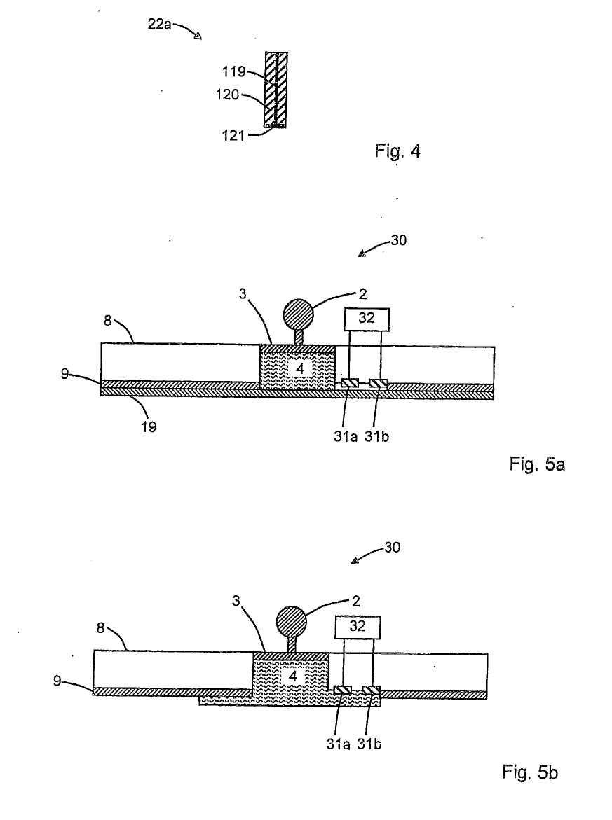

FIG. 4 illustrates a flexible contact of the electrode plug of FIG. 3 in detail;

FIG. 5a illustrates an exemplary embodiment of a medical electrode with a presence sensor for recognizing the presence of the covering;

FIG. 5b shows how the presence sensor of FIG. 5a recognizes the removal of the covering;

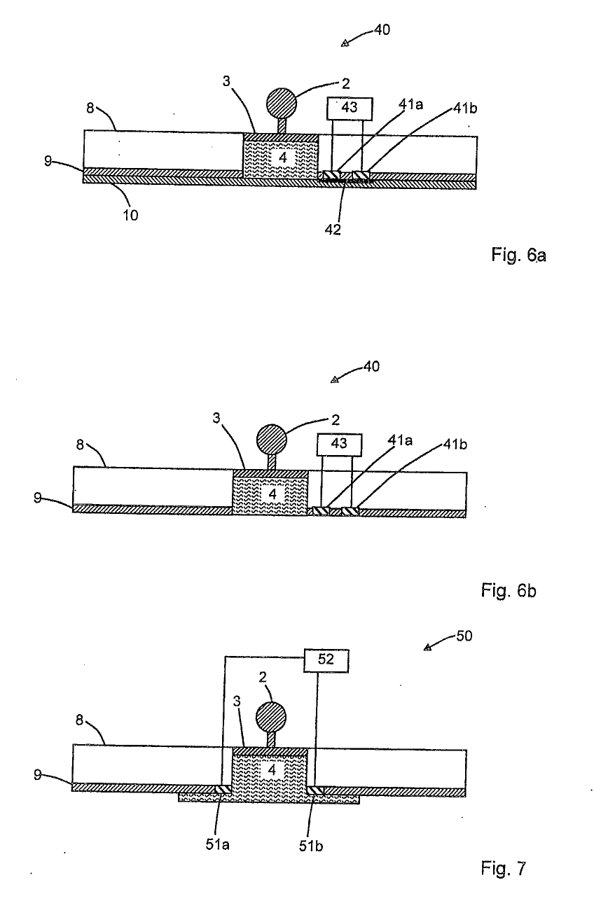

FIG. 6a illustrates an alternative exemplary embodiment of a medical electrode with a presence sensor for recognizing the presence of a covering;

FIG. 6b shows how the presence sensor of FIG. 6a recognizes the removal of the covering;

FIG. 7 shows an exemplary embodiment of a medical electrode with a conductivity sensor for determining the conductivity of the contact means;

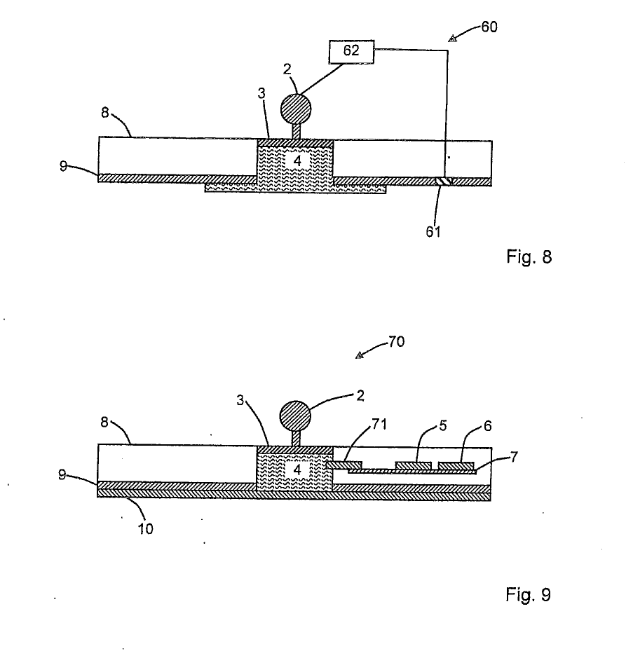

FIG. 8 shows an exemplary embodiment of a medical electrode with a conductivity sensor for determining the transition resistance to the skin; and

FIG. 9 shows an exemplary embodiment of a medical electrode with a chemical sensor for determining a chemical property of the contact means.

FIG. 1 shows a first exemplary embodiment of a medical electrode 1. In the following the same or similar parts of the medical electrode 1 in the description have the same reference numerals and these parts are also described only once in order to avoid repetitions.

The medical electrode 1 in FIG. 1 is constructed as a so-called adhesive electrode and serves to be adhered onto the skin of a patient and to receive bioelectrical signals from the patient. It comprises a metallic head 2 for fastening an electrode cable to an electrode plug. The metallic head 2 has an electrically conductive connection to an electrically conductive electrode plate 3 formed here from metal and serving to receive bioelectrical signals. The metallic head 2 can also be constructed in some exemplary embodiments as a clamp or a clip. The metallic head 2 and the electrical plate 3 are constructed here as two separate elements but can also be constructed in other exemplary examples as one element, e.g., in one piece.

The medical electrode 1 furthermore comprises a contact means 4 which is contained in a hollow space of an electrode body 8 of the medical electrode 1. The contact means 4 is arranged under the electrode plate 3 and serves to establish an electrically conductive connection between the electrode plate 3 and the patient's skin. The contact means 4 is designed here as a gel, as was also explained above.

The electrode body 8 is produced from an electrically insulating material and contains, e.g., plastic and/or textile materials.

The metallic head 2 is arranged of the top of the electrode body 8 so that it is accessible for connecting it to an electrode cable.

An adhesive layer 9 is arranged on the bottom of the electrode body 8 and serves to adhere the medical electrode 1 onto the patient's skin.

The medical electrode 1 comprises on the bottom, i.e., where the adhesive layer 9 is arranged, a covering 10 which protects the adhesive layer 9 and the contact means 4 from drying out and becoming contaminated. Furthermore, the covering 4 ensures that the contact means 4 remains in place and does not run out of its hollow space. The covering 10 is manufactured here from a coated paper but can also be manufactured from plastic or some other suitable material, as a sheet, etc.

The medical electrode 1 is subjected to an ageing process in particular on account of the contact means 4 that is designed here as a gel. The gel can, e.g., dry out or chemically change. Furthermore, the adhesiveness of the adhesive of the adhesive layer 9 can weaken. Therefore, the medical electrode 1 has an expiration date after which a usage can lead to the above-cited losses in the signal quality.

In order to store information about manufacturer, electrode type, charge number and serial number, contact means used and the like in the medical electrode 1, it has an electronic circuit 7 comprising a memory 6 which is designed as a flash memory and can permanently store the information even if no electrical current is being supplied.

Even if the electronic circuit 7 is constructed arranged in the electrode body in the present exemplary embodiment, the present invention is not limited in this regard. In other exemplary embodiments the electronic circuit is arranged outside of the electrode body, e.g., coupled via a conductive connection to the measuring contacts/sensors in the electrode.

The electronic circuit 7 furthermore comprises a microcontroller 5 which is designed to transmit information to the memory 6 and to store it in the memory. The electronic circuit 7 comprises a flexible plate bar on which the memory 6 and the microcontroller 5 are arranged. In other exemplary embodiments the plate bar is designed to be inflexible. The electronic circuit 7 is integrated into the electrode body 8.

Furthermore, the medical electrode 1 comprises a coil 11 on the electronic circuit 7 via which an inductive coupling can take place. Wireless energy and information can be transmitted via the inductive coupling to the electronic circuit 7 and from it to an external device.

In this manner an inductive coupling can be established, for example, as is illustrated in FIG. 2, via an electronic plug 12 of an electrode cable connected to the medical electrode 1.

To this end the electrode plug 12 also comprises a coil 15 which can be connected via two lines 16 and 17 to an external device, e.g., to an EKG device which then also appropriately controls the coil 15 in order to receive information from the direction of transport circuit 7 and/or to transmit information to it. Therefore, the external device can query the electrode-related data from the memory 6.

Furthermore, the electrode plug 12 comprises a metallic coating 13 which has a shape negative to the metallic head 2 so that the electrode plug 12 can be inserted onto the metallic head 2 and the metallic coating 13 can produce an electrical contact with the metallic head 2. The metallic coating 13 makes contact with a line 14 so that the bioelectrical signals received from the electrode plate 3 can be transmitted to the external device.

Consequently, electrical energy and/or information such as the electrode -related data from the memory 6 can be wirelessly transmitted via the inductive coupling of the coil 15 of the electrode plug 12 and of the coil 11 of the medical electrode 1. Even otherwise, a communication between an external electronic system such as an EKG device or an analysis device or the like and the electronic circuit 7 with its connected components is possible. Therefore, e.g., even the sensor of the electronic circuit or a sensor coupled to the electronic circuit can be controlled by the external electronic system via the coupling. Even the memory 6 can be externally controlled in some exemplary embodiments so that in some external embodiments the microcontroller 5 can also be omitted.

In order to guarantee the correct position of the coil 15 and the coil 11 with one another, e.g., a mechanical fixing of the electrode plug 12 can be provided, e.g., by pins, projections, notches, grooves or other intermeshing mechanical means which fix an electrode plug and the medicinal electrode in a defined position to one another. In other exemplary embodiments the coil 11 of the medical electrode 1 is concentrically constructed and extends annularly through the electrode body 8 so that it makes no difference at which location the coil 15 of the electrode plug 12 is arranged since a part of the coil 11 is always located under it. To this end the coil 15 of the electrode plug and the coil 11 of the medical electrode 1 are each arranged with the same distance from the middle axis of the medical electrode 1, which extends centrally through the metallic head 2.

In some exemplary embodiments, instead of or additionally to the coil 11 or 15 a chip for wireless communication, e.g., an RFID chip or the like can be arranged.

In some exemplary embodiments a capacitive coupling is also provided between the electrode plug 12 and the medical electrode 1 which coupling is constructed analogously to the exemplary embodiment of FIG. 2.

A wire-connected coupling between an electrode plug 12′ and a medical electrode 20 is illustrated in FIG. 3.

The medical electrode 20 and the electrode plug 12′ largely correspond to the medical electrode 1 of FIGS. 1 and 2 and to the electrode plug 12 of FIG. 2.

The electronic circuit 7 lacks the coil 11 and instead of it two electrical contacts 21a and 21b are arranged which run through the electrode body 8 toon its surface to the outside.

Accordingly, the electrode plug 12′ comprises two electrical contacts 22a and 22b which electrically contact the electrical contacts 21a and 21b when the electrode plug 12′ is arranged on the medical electrode 20. The electrical contacts 22a and 22b are designed as spring contacts as is shown by way of example in FIG. 4 for the contact 22a. The contact 22a has a rod-shaped section 119 and a plate section 121. The rod-shaped section 119 is surrounded by a spring 120 which tensions the contact 22a in such a manner that the plate section 121 presses against the electrical contacts 21a and 21b of the medical electrode 20 when the electrode plug 12′ is plugged in. The electrical contacts 22a and 22b of the electrode plug 12′ are each connected to the electrical lines 16 and 17 which can be connected to an external device as explained above.

In order to mechanically fix the electrode plug 12′, three magnets 24a, 24b and 24c are arranged on its bottom and opposite them the medical electrode 20 comprises corresponding magnetic metallic elements 23a, 23b and 23c in the top of the electrode body 8. Accordingly, the magnetic attractive force of the magnets 24a, 24b and 24c holds the electrode plug 12′ in its position and it draws it with the bottom to the top of the electrode body 8. As a result, the plate section 121 of the electrical contacts 22a and 22b of the electrode plug 12 is pressed against the electrical contacts 21a and 21b of the medical electrode 1 counter to the spring force of the spring 120, which establishes a good electrical contact.

As a result, electrical energy and/or information such as the electrode-related data can be transmitted via the electrical contacts 21a, 21b, 22a and 22b of the medical electrical 20 and of the electrical plug 12′.

Alternatively, the electrical contacts 21a and 21b of the medical electrical 20 can also be constructed as annular contacts and extend in a corresponding manner as concentric, circular strips on the top of the electrode body 8 in order to ensure the correct position of the spring-supported contacts 22a and 22b of the electrical plug 12′ relative to the electrical contacts 21a and 21b of the medical electrode 20. Furthermore, an additional, mechanical fixing can be present, as explained above.

In the following, other exemplary embodiments of a medical electrode are described, wherein in order to simplify the presentation the electronic circuit and the coupling between an electrode plug and the electronic switch are not shown.

This can be designed, e.g., in a corresponding manner analogously to the exemplary embodiments discussed above, in particular for FIGS. 1 to 3, that is, the medical electrodes described in the following can be constructed for a wireless or wire-connected coupling.

FIGS. 5a and 5b show an exemplary embodiment of a medical electrode 30 in which the removal of the covering 10 can be recognized.

In addition, the medical electrode 30 comprises two electrical contacts 31a and 31b arranged at a distance from one another on its bottom of the electrode body 8. An intermediate space is present between the contacts 31a and 31b so that no current can flow between them. If the covering 10 is removed (FIG. 5b), then the contact means 4 flows out and into the intermediate space between the contacts 31a and 31b, establishing an electrical connection between the two electrical contacts 31a and 31b. Consequently, the contact means 4 closes the two electrical contacts 31a and 31b and forms a transitional resistance that can be determined by a presence sensor 32 which is electrically coupled to the two electrical contacts 31a and 31b.

When the covering 10 is again placed on the electrode carrier 8 the gel of the contact means 4 remains as a thin film and establishes the electrical connection between the electrical contacts 31a and 3 lb and allows it to exist.

The presence sensor 32 is shown here outside of the medical electrode 30 but it can also be a component of the electrical circuit 7 or also be realized by the microcontroller 5 which is arranged in an appropriate manner for determining a current flow or resistance between the two electrical contacts 31a and 31b.

However, the presence sensor 32 can also be provided in an external device or a microprocessor present there can be arranged in an appropriate manner for determining a current flow or resistance between the two electrical contacts 31a and 32b.

FIGS. 6a and 6b show an alternative exemplary embodiment of a medical electrode 40 in which the removal of the covering 10 can be recognized.

To this end the medical electrode 40 has two electrical contacts 41a and 41b arranged at a distance from one another on its bottom of the electrode body 8. The cover 10 comprises a metallic body 42 which can be evaporated on can be constructed as a metallic sheet, as a metallic strip or the like and is arranged so that it electrically connects the two electrical contacts 41a and 41b to one another.

If the covering 10 is removed (FIG. 6b), the electrical contact between the two electrical contacts 41a and 41b is interrupted. This interruption of the current flow can be determined by a presence sensor 42 which is electrically coupled to the two electrical contacts 41a and 41b.

The presence sensor 42 is shown here outside of the medical electrode 40 but it can also be a component of the electronic circuit 7 or also be realized by the microcontroller 5, which is appropriately designed to determine a current flow or resistance between the two electrical contacts 41a and 41b.

However, the presence sensor 42 can also be provided in an external device or a microprocessor present there can be appropriately designed to determine a current flow or resistance between the two electrical contacts 41a and 41b.

In other exemplary embodiments the presence sensor comprises a pin which is drawn with the covering out of the electrode, as a result of which a contact is opened or closed. As a result, it can be determined whether the cover is present or not.

FIG. 7 shows an exemplary embodiment of a medical electrode 50 in which the conductivity of the contact means 4 is determined by measuring an electrical resistance or in impedance between two electrical contacts 51a and 51b. The electrical contacts 51a and 51b are arranged at a distance from one another, wherein the contact means 4 establishes an electrical contact between them. The electrical contacts 51a and 51b can be arranged in such a manner that the contact means 4 is arranged between them when the covering 10 is present and/or when it is removed. In some exemplary embodiments even the electrical contacts 31a and 31b can be used for measuring the conductivity which was explained above for checking the presence of the covering 10.

The resistance and/or the impedance between the two electrical contacts 51a and 51b is determined by measuring a direct current and/or an alternating current which is performed by a conductivity sensor 52 which is electrically coupled to the two electrical contacts 51a and 51b.

The conductivity sensor 52 is shown here outside of the medical electrode 50 but it can also be a component of the electrical circuit 7 or also be realized by the microcontroller 5, which is appropriately designed to determine a current flow or resistance between the two electrical contacts 51a and 51b.

However, the conductivity sensor 52 can also be provided in an external device or a microprocessor present there can be appropriately designed to determine a current flow or resistance between the two electrical contacts 51a and 51b.

FIG. 8 shows an exemplary embodiment of a medical electrode 60 in which a transitional resistance is determined between the medical electrode 60 and the skin of a patent by measuring an electrical resistance or an impedance between an electrical contact 61 and the electrode plate 3.

The electrical contact 61 is arranged on the bottom of the electrode body 8 so that it comes in contact with the skin of the patient when the medical electrode 60 is adhered fast with it adhesive layer 9.

The resistance or the impedance between the electrode plate 3 and the electrical contact 61 is determined by a direct- and/or alternating current measuring which is carried out by a conductivity sensor 62 which is electrically coupled to the electrode plate 3 via the metallic head 2 and to the electrical contact 61.

The conductivity sensor 62 is shown here outside of the medical electrode 50 but it can also be a component of the electronic circuit 7 or also be realized by the microcontroller 5, which is appropriately designed to determine a current flow or resistance between the electrode plate 3 and the electrical contact 61.

However, the conductivity sensor 62 can also be provided in an external device or a microcontroller present there can be appropriately designed to determine a current flow or resistance between the electrode plate 3 and the electrical contact 61.

FIG. 9 shows an exemplary embodiment of a medical electrode 70 in which a chemical property of the contact means 4 is determined by a chemical sensor 71 which is a component of the electronic circuit 7 and is connected to the microcontroller 5. Chemical sensors are basically known and an appropriate chemical sensor can be selected depending on what type of property of the contact means 4 is to be determined.

The exemplary embodiments of FIGS. 5a to 7 show two electrical contacts and only one contact is shown in the exemplary embodiment of FIG. 8. However, the present invention is not limited to a certain number of electrical contacts.

For the rest, the microcontroller 5 or the memory 6 can be designed to store data stemming from one of the above sensors 32, 43, 52, 62, 71 in the memory 6.

1. A medical electrode for receiving bioelectrical signals of a muscle via the skin of a person or of an animal, comprises:
a metallic contact for the connection to an electrode cable;
an electrically conductive electrode plate for receiving the bioelectrical signals;
a contact means for establishing an electrical contact between the electrode plate and the skin; and
an electronic circuit comprising a memory for storing electrode-related data.

2. The medical electrode according to claim 1, wherein the electronic circuit is designed to transmit the electrode-related data to a receiver.

3. The medical electrode according to claim 2, wherein the electronic circuit is designed to wirelessly transmit the electrode-related data.

4. The medical electrode according to claim 3, wherein the electronic circuit comprises a transponder and is designed to transmit by radio the electrode-related data as a reaction to a received radio signal.

5. The medical electrode according to claim 2, also comprising an electrode body in which the electronic circuit is arranged, wherein electrical contacts are run from the electronic circuit to an upper side of the electrode body.

6. The medical electrode according to claim 1, also comprising a covering on the contact means and a presence sensor, wherein the presence sensor is designed to recognize whether the covering is present.

7. The medical electrode according to claim 1, also comprising a conductivity sensor designed to measure the electrical conductivity of the contact means.

8. The medical electrode according to claim 1, also comprising a conductivity sensor designed to measure the conductivity between the electrode plate and the skin.

9. The medical electrode according to claim 1, also comprising a chemical sensor designed to determine a chemical property of the contact means.

10. The medical electrode according to claim 1, wherein the electronic circuit is designed to store measured data from a sensor of the medical electrode in the memory.
